(19) United States
(12) Patent Application Publication
(10) Pub. No.: US 2018/0250156 A1
(43) Pub. Date: Sep. 6, 2018

(54) Kit for an ostomy device

(71) Applicant: Coloplast A/S, Humlebaek (DK)

(72) Inventor: Peter Kwok Hing Lam, Frederiksberg C (DK)

(21) Appl. No.: 15/559,711

(22) PCT Filed: Mar. 17, 2016
(86) PCT No.: PCT/DK2016/050077

§ 371 Date: Sep. 19, 2017

(30) Foreign Application Priority Data
Mar. 20, 2015 (DK) PA 201570159
Aug. 28, 2015 (DK) PA201570561

(51) Int. Cl. A61F 5/443

(52) U.S. Cl. CPC A61F 5/443; A61F 5/445

(57) ABSTRACT

Disclosed is a method of attaching an adhesive, such as an adhesive on an adhesive base plate of an ostomy device, to a skin surface of a user. The method involves the steps of providing an adhesive, for instance on a base plate of an ostomy device. The adhesive can be disposed on a skin-facing surface of a backing layer. Also provided is a solvent capable of at least partly dissolving the adhesive. The solvent is brought into contact with the adhesive and the adhesive is applied to the skin of the user. Also disclosed is a kit of parts including an adhesive and a solvent capable of at least partly dissolving the adhesive.

Disclosed is a method of attaching an adhesive, such as a base plate of an ostomy device, to a skin surface of a user.

### BACKGROUND

In connection with surgery for a number of diseases in the gastro-intestinal tract, one of the consequences in many cases is that the patient is left with an abdominal stoma, such as a colostomy, an ileostomy, or a urostomy, in the abdominal wall for the discharge of visceral contents. The discharge of visceral contents cannot be regulated at will. For that purpose, the user will have to rely on an appliance to collect the material emerging from such opening in a bag, which is later emptied and/or discarded at a suitable time. Ostomy devices are typically attached to the skin of the ostomy user by means of an adhesive wafer, also termed a baseplate, on the ostomy appliance.

### BRIEF DESCRIPTION OF THE DRAWINGS

FIGS. 1-4 show embodiments of an ostomy base plate comprising multiple release liners.

### DETAILED DESCRIPTION OF THE INVENTION

Embodiments provide a method of attaching an adhesive base plate of an ostomy device to a skin surface of a user, comprising the steps of

a) providing an adhesive base plate comprising a backing layer and an adhesive;

b) providing a solvent capable of at least partly dissolving the adhesive;

c) bringing the solvent into contact with the adhesive; and

d) applying the adhesive to the skin, thereby attaching the adhesive base plate to the skin surface of the user.

The method may be carried out by a person other than the user to which the adhesive is attached. For instance, the method may be carried out by a commercial service provider assisting the user for a fee. Such commercial service providers exist and provide fee-based services to, e.g., ostomy users or people with wounds. The service may include the service provider removing and applying ostomy bags for the ostomy user or removing and applying wound dressings for a person with wounds.

The method may also be carried out in order to obtain a sample of the output from the ostomy user or wound exudate from the person with wounds. For instance, a healthcare professional may require a stoma output or wound exudate sample in order to make medical decisions or generally assess the physical state of a user. In such cases, the healthcare professional may order the sampling to be done by a professional service provider to ensure that the sampling happens correctly. Again, a fee-based commercial service provider would carry out the method with the aim of providing a sample to the healthcare professional. Such paid services exist on commercial terms and operate on a continuous and independent basis with an aim of financial gain. They are not exclusively dependent for their operation on the instructions of the user in question. For instance, they may work directly under the instructions of a healthcare professional.

Embodiments provide a method of attaching an adhesive base plate of an ostomy device to a skin surface of a user, comprising the steps of

a) providing an adhesive base plate comprising a backing layer and an adhesive disposed on a skin-facing surface of the backing layer;

b) providing a solvent capable of at least partly dissolving the adhesive;

c) bringing the solvent into contact with the adhesive; and

d) applying the adhesive to the skin, thereby attaching the adhesive base plate to the skin surface of the user.

A challenge facing all ostomy users is to get the adhesive of the ostomy device adhesive base plate to adhere properly to the skin of the stomach surrounding the stoma. This is relevant when initially applying the adhesive base plate to the skin, and also during wear if the adhesive base plate starts to lose adhesion and partly or fully detach from the skin. An insufficient adhesive bond between the adhesive base plate and the skin can lead to leakage of ostomy output into the adhesive and onto the skin. This can cause serious skin problems as well as practical problems for the user.

The method comprises the step of bringing a solvent into contact with the adhesive of the adhesive base plate. The purpose of this is to at least partly dissolve the adhesive. The present inventor found that the at least partial dissolution of the adhesive actually improves the ability of the adhesive to form a quick and strong adhesive bond with the skin surface. This is contrary to the accepted wisdom within the field, which considers solvents as useful for destroying and/or removing adhesives or adhesive residues from the skin.

By way of explanation only, what is thought to happen is that the solvent starts to dissolve the adhesive layer from the surface to the bulk of the adhesive polymer, changing the rheology by, e.g., reducing the moduli. At a molecular level, the individual polymer chains gain a greater degree of movement, which, when applied to the skin, allows for fast wetting of the surface in that there is sufficient flow into the structures of the skin to quickly establish a large contact area and a strong bond, both in terms of mechanical anchoring and adhesion.

The present inventor has found that a solvent that at least partly dissolves the adhesive will significantly increase the tack and decrease the cohesion of the adhesive. The increase in tack is enough to ensure that a good adhesive bond quickly forms between the adhesive base plate and the skin. The lowered cohesion likely contributes to this by making the adhesive softer and more pliable and thereby better able to quickly flow into the micro- and macro-structures of the skin.

The direct effects of the presence of solvent decrease or wear off over time due to evaporation of the solvent. However, the adhesive bond formed will remain stronger than it would otherwise have been because the initial strong adhesion and low cohesion has ensured a very good bond, both mechanical and adhesive, between the skin and the adhesive. The cohesion of the adhesive will also increase to its normal level as the solvent evaporates.

Generally, solvent will diffuse through and evaporate from the adhesive via the non-skin-facing side, or through the edges. This will reduce or eliminate the solvent and its effect on the surface of the adhesive in contact with the substrate.

The normal, or un-dissolved, properties of the adhesive are the properties of the adhesive before it is brought into contact with the solvent and/or after the solvent has evaporated from the adhesive, for instance to a level where less than 1% of the solvent remains.

The solvent will take some time to solubilize the adhesive and will also take some time to evaporate from the adhesive. Generally, the properties of the adhesive change gradually as the solvent enters and solubilizes the adhesive and also as the solvent gradually evaporates. The time from when the adhesive is first brought into contact with the solvent to when the adhesive is maximally dissolved is termed the dissolution time. This time will be different for different solvent-adhesive combinations and will also depend on the volume of the involved compositions as well as, at least, temperature, ventilation, surface area, and humidity. In embodiments, the dissolution time is less than 5 minutes, such as less than 2 minutes, such as less than 1 minute, such as less than 30 seconds, such as less than 10 seconds, such as less than 1 second. In embodiments, the dissolution time is 1-10 seconds, 1-30 seconds, or 10-30 seconds. The shorter the dissolution time, the faster the adhesive will be maximally dissolved and thus have attained the properties caused by the dissolution, such as high tack and lower cohesion and/or viscosity. A short dissolution time will therefore make it possible for the adhesive to quickly attain the beneficial properties caused by the dissolution.

Similarly, the time from when the solvent is first brought into contact with the adhesive to when less than 1% of the solvent remains in contact with the adhesive is termed the evaporation time. In embodiments, the evaporation time is 1-10 minutes, 1-5 minutes, 1-2 minutes, less than 1 minute, less than 30 seconds, at least 10 seconds, at least 30 seconds, at least 1 minute, or at least 5 minutes. In embodiments, the solvent has an evaporation time that is lower than that of water. A low evaporation time will ensure that the adhesive returns to its normal, un-dissolved properties quickly. For instance, a low evaporation time will ensure that the cohesion of the adhesive quickly returns to normal, thus ensuring that the temporary low cohesion caused by the dissolution does not negatively affect the ability of the adhesive to remain securely attached and maintain its integrity. Evaporation time is inversely proportional to evaporation rate.

In embodiments, the solvent is applied prior to the first attachment of the adhesive base plate to the skin of the user. This means that step (c) occurs before step (d). In this way, the adhesive can be activated by the solvent prior to attachment of the adhesive base plate to the skin, thus ensuring a strong and durable adhesive bond once the adhesive is attached.

When applying the solvent prior to or at the same time as the initial attachment of the adhesive base plate to the skin, an adhesive with an initial lower adhesion can be used. This is because the adhesive will be activated by the solvent, causing it to gain a sufficiently high adhesion at the time of application. Similarly, an adhesive with a higher cohesion than normal may be used because the solvent will ensure a proper low cohesion at the time of application of the adhesive to the skin. It is advantageous to use a relatively low-adhesion adhesive because such an adhesive will be easier to handle, during both production and use. Also, dirt and other unwanted material will be less likely to stick to the adhesive and potentially weaken the adhesive bond formed. An adhesive with a relatively high cohesion prior to application of the solvent is advantageous in that such an adhesive will more easily maintain its integrity, both during storage and use, and will also be easier to remove from the skin without leaving adhesive residue. An adhesive with an adhesion and cohesion within the normal ranges for ostomy adhesives may also be improved by the instant method in that an increased initial tack is still provided by the solvent activation.

In embodiments, the solvent is used to re-activate the adhesive after the adhesive base plate has been attached to the skin. This means that step (c) occurs after step (d) or that step (c) occurs both before and after step (d). In this case, the solvent is applied to at least a part of the adhesive that has detached from the skin. The detachment may have happened because of poor adhesion or long-term use, or it may be that the user has on purpose detached a part of the adhesive from the skin in order to apply or re-apply the solvent to ensure a stronger bond. The solvent is then applied to the detached part of the adhesive and the adhesive is re-attached to the skin. Again, the improved adhesion and decreased cohesion caused by the solvent will result in the adhesive regaining a strong bond to the skin. The application of the solvent to an adhesive that has already been adhered to the skin for some time is referred to as “re-activation” of the adhesive.

The solvent may be applied only prior to the initial attachment of the adhesive to the skin or only after the initial attachment or both before initial attachment and after initial attachment.

In embodiments, the solvent is applied to the adhesive of the adhesive base plate. In this way, the solvent can remove dirt and other material that has adhered to the adhesive, thus cleaning the adhesive and making sure such dirt and material does not interfere with the adhesion.

In embodiments, the solvent is applied to the skin surface of the user. The adhesive is then subsequently applied to the skin, which still has the solvent on it. Applying the solvent to the skin will help in cleaning the skin prior to attachment of the adhesive, thus improving adhesion. After application of the solvent to the skin, the adhesive is applied to the skin, which still has the solvent on its surface. In some embodiments, an excess of solvent is applied to the skin so that some evaporation can occur prior to attachment of the adhesive, while still leaving enough solvent on the skin surface to at least partly dissolve the adhesive.

As mentioned, the solvent is further advantageous in that it can effectively clean the adhesive surface and/or the skin by removing dirt and other unwanted material. This will improve adhesion and minimize the disruption caused by such material between the adhesive and the skin.

In embodiments, the adhesive base plate further comprises a release liner disposed on the skin-facing surface of the adhesive, and the method comprises the further step of removing the release liner from the adhesive, performed prior to step (c). The release liner will protect the adhesive during storage and handling prior to application.

In embodiments, step (c) occurs before step (d). This means that the solvent is brought into contact with the adhesive prior to the adhesive being applied to the skin. In this way, the adhesive is activated prior to the first application of the adhesive to the skin.

In embodiments, step (c) occurs after step (d). This means that the solvent is brought into contact with the adhesive after the adhesive has been applied to the skin. In this way, the adhesive is re-activated after already having been attached to the skin for some time.

In embodiments, step (c) occurs at the same time as step (d). In this way, the adhesive comes into contact with the solvent at the same time as it comes into contact with the skin. This can be brought about by the solvent having been applied to the skin.

In embodiments, the adhesive comprises a polar polymer. In embodiments, the adhesive comprises a hydrophilic polymer. In embodiments, the adhesive comprises a non-polar polymer. In embodiments, the adhesive comprises a hydrophobic polymer. In embodiments, the adhesive comprises a polymer selected from the group consisting of silicone, styrene block copolymer, polyurethane, acrylate, and ethylene block copolymer. In embodiments, the adhesive comprises a polymer selected from the group consisting of styrene-isoprene-styrene (SIS), ethylene vinyl acetate (EVA), styrene-butadiene-styrene (SBS), butyl rubber, polyisobutylene (PIB), polyether, polyester, polyurea, and epoxy-based polymer. In embodiments, the adhesive comprises a hydrid or physical mixture of two or more of said polymers. In embodiments, the adhesive comprises a homopolymer, a random copolymer, a block copolymers, a straight-chained polymer, a branched polymer, or a dendrimer.

In embodiments, the adhesive is a thermoplastic adhesive. In embodiments, the adhesive is physically cross-linked and/or covalently cross-linked and/or ionically cross-linked. In embodiments, the adhesive comprises particles, fibres, absorbent materials, fillers, tackifiers, oils, and/or plasticizers.

In embodiments, the adhesive comprises absorbent material. In embodiments, the absorbent material is present in an amount of 1-60% (w/w) of the adhesive.

For instance, the adhesive comprises a water absorbent material in an amount of 1-40% (w/w) or 1-20% (w/w) or 20-40% (w/w) or 20-60% (w/w) or 40-60% (w/w) or 25-50% (w/w) of the adhesive.

In some embodiments, the water absorbent material is selected from hydrocolloid, water soluble salt, mono, di- and oligosaccharides, sugar alcohols, polypeptides, organic acids, inorganic acids, amino acids, amines, urea, super absorbent particles such as polyacrylic acid, glycols such as polyethylene glycol, fumed silica, bentone, bentonite, and mixtures thereof.

In some embodiments, the hydrocolloid is selected from guar gum, locust bean gum, pectin, potato starch, alginates, gelatine, xantan or gum karaya, cellulose derivatives, salts of carboxymethyl cellulose, sodium carboxymethyl cellulose, methyl cellulose, hydroxypropyl cellulose, hydroxyethyl cellulose, sodium starch glycolate, polyvinylalcohol, and mixtures thereof.

In some embodiments, the water soluble salt is selected from NaCl, CaCl₂, K₂SO₄, NaHCO₃, Na₂CO₃, KCl, NaBr, NaI, KI, NH₄Cl, AlCl₃, CH₃COONa, CH₃COOK, HCOONa, HCOOK, and mixtures thereof.

In embodiments, the solvent is a polar solvent. In embodiments, the solvent is a hydrophilic solvent or a non-polar solvent or a hydrophobic solvent. In embodiments, the solvent is selected from the group consisting of silicone fluid, ethyl acetate, acetone, tetrahydrofuran (THF), butyl acetate, hexane, ethanol, iso-propanol, dichloromethane, methanol, acetonitrile, toluene, dimethylformamide (DMF), dimethyl sulfoxide (DMSO), pyridine, cyclohexane, and n-butanol. In embodiments, the chosen solvent or combination of solvents depend on the desired evaporation rate and effect.

In embodiments, a mixture of different solvents is used. In embodiments, the solvent comprises one or more additional ingredients selected from the group consisting of polymers, surfactants, and mass transport promoters. A mass transport promoter is a compound or composition that will increase the evaporation rate of the solvent.

In embodiments, the adhesive is a silicone pressure sensitive adhesive and the solvent is silicone fluid, hexamethyldisiloxane (HMDS), or ethyl acetate. In embodiments, the adhesive is a styrene copolymer pressure sensitive adhesive, such as a mixture of SIS and polyisobutylene, and the solvent is acetone or THF or ethyl acetate or butyl acetate or hexane. In embodiments, the adhesive is a polyurethane pressure sensitive adhesive and the solvent is THF. In embodiments, the adhesive is an acrylate pressure sensitive adhesive and the solvent is ethanol or iso-propanol. In embodiments, the adhesive is an EVA pressure sensitive adhesive and the solvent is THF or iso-propanol. These are just examples of possible combinations of polymer and solvent types. Other combinations are possible.

Various physical parameters of the adhesive may be changed by activating or re-activating the adhesive by bringing it into contact with the solvent. In embodiments, a property of the adhesive selected from the group consisting of tack and peel force is increased after activation or re-activation. In embodiments, a property of the adhesive selected from the group consisting of viscosity, modulus, shear force, and cohesion is decreased after activation or re-activation.

In embodiments, the solvent is provided in the form of a cloth or non-woven material, such as a wet wipe or a cloth or a sponge or a cotton swab, which has been wetted with the solvent prior to use. In embodiments, the solvent is provided in a container, such as a solvent in a spray.

In embodiments, the solvent is applied by wiping, spraying, painting, dripping, rolling, squirting, brushing, or dipping.

In embodiments, the volume of solvent brought into contact with the adhesive is 0.01-5 ml, 0.01-4 ml, 0.01-3 ml, 0.01-2 ml, 0.01-1 ml, 0.1-5 ml, 0.1-4 ml, 0.1-3 ml, 0.1-2 ml, 0.1-1 ml, 0.5-5 ml, 0.5-4 ml, 0.5-3 ml, 0.5-2 ml, 0.5-1 ml, 1-5 ml, 1-4 ml, 1-3 ml, 1-2 ml, at least 0.01 ml, at least 0.1 ml, at least 1 ml, less than 5 ml, less than 4 ml, less than 3 ml, less than 2 ml, less than 1 ml, or less than 0.1 ml.

In embodiments, the volume of solvent is less than 30%, such as less than 20%, such as less than 10%, such as less than 5%, such as less than 1%, such as less than 0.1%, such as less than 0.01%, of the volume of the adhesive. In embodiments, the volume of the solvent is 0.01-0.1%, 0.01-1%, 0.1-1%, 1-5%, 1-10%, 5-10%, 10-20%, 10-30%, or 20-30% of the volume of the adhesive.

In embodiments, the adhesive is an adhesive layer. In embodiments, the adhesive layer has a thickness of 30-5,000 micrometers, 50-2,000 micrometers, 100-1,000 micrometers, at least 30 micrometers, at least 50 micrometers, at least 100 micrometers, less than 200 micrometers, or less than 100 micrometers. In embodiments, the adhesive comprises more than one layer of adhesive, wherein the total thickness of the adhesive layers is as indicated above. In embodiments, the adhesive comprises a pattern, or holes, or dots.

In embodiments, the backing layer (sometimes also referred to as a top film or a carrier film) is a film, a foam, a textile, or a non-woven. The backing layer may be moisture permeable. In embodiments, the backing layer has a thickness of 10-2,000 micrometers, 50-1,000 micrometers, 100-500 micrometers, or 10-100 micrometers. In embodiments, the backing layer is thermoplastic, which will enable heat welding of the backing layer.

Embodiments provide a kit of parts comprising an adhesive base plate for an ostomy device, the adhesive base plate comprising an adhesive as defined herein, and the kit further comprising a solvent capable of at least partly dissolving the adhesive. The adhesive base plate may comprise a backing layer with the adhesive disposed on a skin-facing surface of the backing layer. The solvent may be a solvent as defined herein. By providing a kit of parts, the user is provided in a single kit with both the adhesive base plate and the solvent that is to be brought into contact with the adhesive of the base plate. This makes it easier for the user to practise the method of attaching the adhesive base plate to the skin and it ensures that the user is applying a solvent that is capable of at least partly dissolving the specific adhesive provided on the adhesive base plate.

Embodiments provide a method of attaching an adhesive to a skin surface of a user, comprising the steps of

a) providing an adhesive;

b) providing a solvent capable of at least partly dissolving the adhesive;

c) bringing the solvent into contact with the adhesive; and

d) applying the adhesive to the skin, thereby attaching the adhesive to the skin surface of the user.

In embodiments, the adhesive is disposed on a skin-facing surface of a backing layer. In embodiments, the adhesive is an adhesive on a wound care product, such as a wound dressing. In embodiments, the method is a method of attaching a wound care product or a medical device comprising the adhesive to a skin surface of a user. In embodiments, the medical device is a sensor patch, an ostomy device, or an external catheter, such as a urisheath.

Embodiments provide a kit of parts comprising an adhesive and a solvent capable of at least partly dissolving the adhesive. The adhesive may be an adhesive as defined herein, The solvent may be a solvent as defined herein. In embodiments, the adhesive is an adhesive on a wound care product or a medical device as defined herein.

In embodiments, the medical device is an ostomy device or a wound dressing comprising at least one release liner disposed on the adhesive. In embodiments, the ostomy device or wound dressing comprises at least two, such as two, or at least three, such as three, or at least four, such as four, release liners. The release liners may be non-overlapping, partly overlapping, or completely overlapping.

In embodiments, the ostomy device or wound dressing comprises two release liners, wherein one release liner covers a central part of the adhesive and another release liner covers a periphery of the adhesive. The central release liner may be arranged concentric to a central aperture, such as in an ostomy adhesive wafer.

In embodiments, the ostomy device or wound dressing comprises three release liners. The release liners may be in the form of two side release liners covering the side portions of the wafer and a central liner covering the middle part of the wafer. One of the release liners may cover the central portion of the adhesive surface. The central liner may be arranged concentric to a central aperture, such as in an ostomy adhesive wafer, and the rest of the adhesive surface may be covered by two side release liners.

The release liner system may be symmetric over the adhesive surface or it may be asymmetric. In embodiments, the release liner is in the form of a polymer film, foil or paper, preferably having release properties that enable the adhesive to be released easily from the liner. Such properties may be inherent in the material or the layer may be siliconized or coated with a low surface tension coating. In general, release liners are made on a mechanically stiff backing such as paper, polyethylene, polypropylene, or polyethylene terephthalate. The stiffness will support the adhesive wafer when applying the collecting device. Multiple separate release liners may be made of the same or different materials.

In embodiments, the ostomy device or wound dressing comprises two or more different adhesives. In embodiments, the ostomy device or wound dressing comprises two or more zones comprising different adhesives. In embodiments, the ostomy device comprises two zones with different adhesives. In embodiments, the ostomy device or wound dressing comprises three zones, wherein two of the zones comprises the same adhesive and the third zone comprises a different adhesive.

In embodiments, separate release liners cover the different adhesives and/or the different zones of adhesive. If not all of the adhesive surface is to be brought into contact with the solvent, it is advantageous to provide separate release liners for the adhesive that is to be brought into contact with the solvent and the adhesive that is not. For instance, a central part of the adhesive wafer, such as a ring-shaped zone adjacent to and surrounding a central aperture in an ostomy adhesive base plate, may be meant to be brought into contact with a solvent, while the remainder of the adhesive wafer is not meant to be brought into contact with the solvent. As such, a separate release liner is provided for the central part and one or more separate release liners are provided for the remaining parts. Alternatively, the opposite may apply: A peripheral zone of the adhesive base plate is to be brought into contact with solvent, whereas a central zone is not. In that situation, a similar arrangement of release liners may be preferred, but the sequence of removing the release liners may be different to allow application of solvent to only chosen zones of adhesive.

In use, the one or more release liners covering adhesive that is to be brought into contact with solvent may be removed first. Then solvent may be applied to the exposed adhesive. Afterwards, the adhesive wafer may be applied to the skin and the remaining release liners removed. Or, alternatively, the remaining release liners may be removed prior to arranging the adhesive wafer on the skin. It may be advantageous, in terms of handling, to only remove a central release liner first, and then apply the adhesive wafer to the skin before removing the remaining release liner(s). In this way, the remaining release liner(s) may support and stiffen the adhesive wafer during application to the skin, thus making it easier to handle and to avoid wrinkles or folds in the adhesive wafer.

In embodiments, after having been brought into contact with the solvent the adhesive has a peel force, measured as described herein at 30 minutes dwell time or at 24 hours dwell time, of at least 1.5 time, such as 2 times, the peel force of an identical adhesive not having been brought into contact with the solvent.

Embodiments provide a method of attaching an adhesive base plate of an ostomy device to a skin surface of a user, comprising the steps of

a) providing an adhesive base plate comprising a backing layer and an adhesive;

b) providing a solvent capable of at least partly dissolving the adhesive;

c) bringing the solvent into contact with the adhesive, thereby increasing the peel force by at least 50%, such as by at least 100% compared to the adhesive not having been brought into contact with the solvent; and

d) applying the adhesive to the skin, thereby attaching the adhesive base plate to the skin surface of the user.

In embodiments, the peel force is measured as described herein. In embodiments, the peel force is measured after 30 minutes dwell time. In embodiments, the peel force is measured after 24 hours dwell time. In embodiments, the peel force is increased by at least 50%, such as by at least 100% compared to the adhesive not having been brought into contact with the solvent in the 30-minute dwell time measurement and/or in the 24-hour dwell time measurement.

### DETAILED DESCRIPTION OF THE DRAWINGS

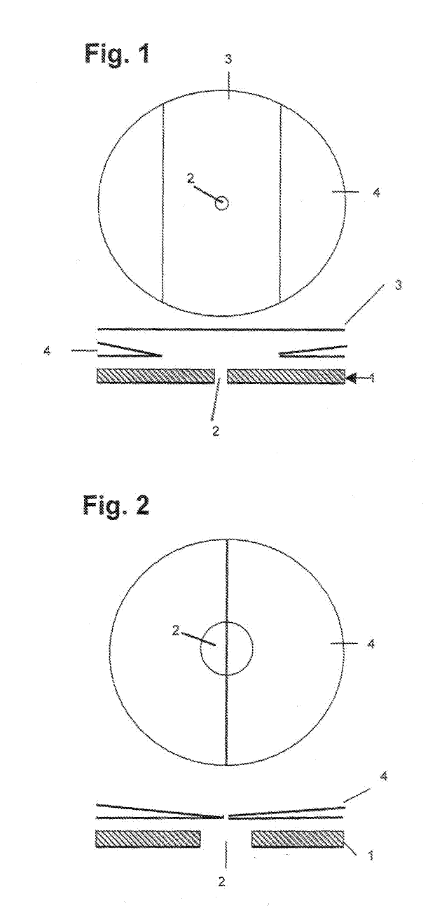

FIGS. 1-4 show adhesive base plates with multiple release liners. Each Figure shows the base plate seen from the skin-facing side and in an exploded cross-section. The base plate is shown without attached coupling means or collection pouch mounted in the non-skin-facing surface of the wafer.

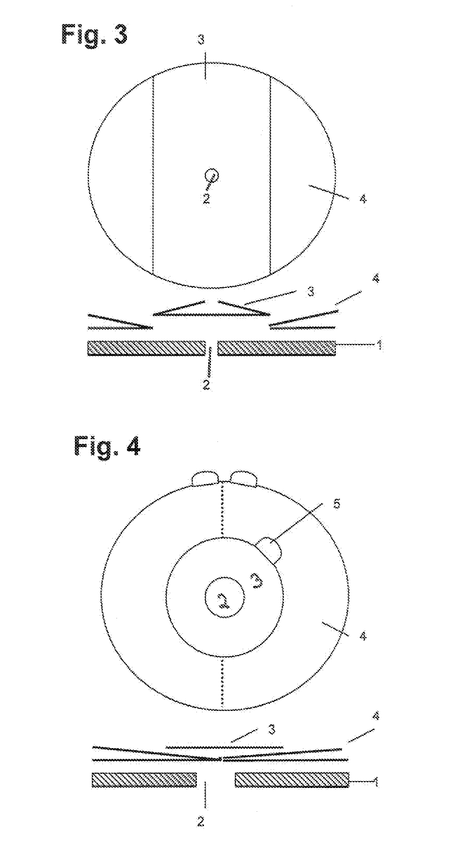

In FIG. 1 is shown a base plate (1) with a central aperture (2) for receiving a stoma, the wafer being provided with a three-split release liner system, comprising two side liners (4) covering the side portions of the adhesive surface and the third middle release liner (3) covering the middle portion of the adhesive wafer. By middle portion is meant the area from diametrical edge to edge and including the central portion of the wafer. The side release liners (4) are optionally folded in order to ease grip and detachment. The release liners can optionally be provided with an ear for easy removal. The middle liner may overlap partly or fully some or all of the side release liners and may have folded edge portions, as shown in FIG. 3, to facilitate easy grip.

In FIG. 2 is shown a two-split release liner system where two side release liners (4) each cover half of the adhesive surface of the base plate (1). The liners are preferably folded to provide easy grip. For application, the user may pull one or both lines away from the aperture, gradually exposing the adhesive surface and attaching said surface to the skin. The wafer is easily handled and applied as the adhesive is exposed gradually and the remaining part still has stiffness from the remaining part of the release liners. When the unprotected part of the skin-facing adhesive is placed on the skin, the rest of the release liners are rolled off and removed.

FIG. 3 shows a three-split release liner wherein all three release liners are folded and wherein there is no overlap of the release liners.

FIG. 4 shows an embodiment where the base plate (1) is provided with side release liners (4) and a central liner (3) covering the central part of the wafer. By the central part is meant the area surrounding the aperture (2). The separately removable central release liner (3) may be in the form of a ring, oval or angular (e.g. triangle, square, or pentangle) shape intended for removal first. The release liners in FIG. 4 are shown with ears (5) for easy grip. The wafer is then fastened to the skin around the soma and subsequently the side release liners may be rolled off. In embodiments, the two side release liners, as illustrated in FIG. 4, are formed as a single release liner by not including the split marked with dotted lines at the bottom of the release liner. The top split, also marked with dotted lines, may still be included to make it easier to remove the release liner, for instance when a collecting pouch is attached to the base plate. Alternatively, both the top and the bottom splits, marked with dotted lines in FIG. 4, may be left out to form one single rim release liner covering the periphery of the adhesive base plate.

In embodiments, each release liner covers a different zone of adhesive. In embodiments, the different zones of adhesive may comprise different adhesives. For instance, one or more zones may comprise adhesive that is to be brought into contact with solvent and one or more zones may comprise adhesive that is not to be brought into contact with solvent.

For instance, in FIG. 4, the adhesive under the central release line (3) may be an adhesive that is to be brought into contact with solvent, whereas the adhesive under the other release liners is not meant to be brought into contact with solvent. In use, the central release liner (3) is removed first, solvent is applied to the adhesive underneath, and the other release liners are then removed afterwards, either before or after the solvent-treated central adhesive has been adhered to the skin.

### EXAMPLES AND METHODS

Measurement Methods

Shear

For measuring shear, the adhesive is laminated on a backing film of the required thickness and cut to a sample of 25 mm wide, 25 mm long.

The sample is stuck abeam to an object glass about 5 mm from the end of the glass, via a double-sided adhesive tape on the film face. A brass block of specified weight is placed on top of the adhesive with a light pressure, with the top of the block level with the top edge of the adhesive. The object glass is fastened with the adhesive and the block vertically on a frame.

The vertical displacement is measured with a calibrated ruler after 24 hrs, or note the time for total detachment of the block if less. The latter may be facilitated by a series of still photos taken at timed intervals.

Dynamic Mechanical Analysis (DMA)

The DMA to measure such parameters as G′, G″, tan(δ), and complex viscosity |η*|, was carried out as follows by a frequency sweep. An adhesive sheet of defined thickness is used. A round sample of 25 mm in diameter was cut out and placed in a Haake RheoStress 6000 rotational rheometer from Thermo Scientific. The geometry applied was parallel plates 25 mm and the shear stress was fixed at 5556 Pa. The measurements were carried out at 32° C.

For the complex viscosity |η*| the value measured at a frequency of 0.01 Hz was used.

MVTR

Moisture vapour transmission rate (MVTR) is measured in grams per square meter (g/m²) over a 24 hours period using an inverted cup method.

A container or cup that was water and water vapour impermeable having an opening of 035 mm was used. 20 mL saline water (0.9% NaCl in demineralised water) was placed in the container and the opening was sealed with the test adhesive mounted on a highly permeable polyurethane (PU) backing film (BL9601 foil from Intellicoat). The container was placed into an electrically heated humidity cabinet and the container or cup was placed upside down, such that the water was in contact with the adhesive. The cabinet was maintained at 32° C. and 15% relative humidity (RH).

The weight loss of the container was followed as a function of time. The weight loss was due to water transmitted through the adhesive and/or film. This difference was used to calculate the MVTR of the test adhesive film. MVTR was calculated as the weight loss per time divided by the area of the opening in the cup (g/m²/24 h).

The MVTR of a material is a linear function of the thickness of the material. Thus, when reporting MVTR to characterize a material, it is important to inform about the thickness of the material for which MVTR was reported. We used 150 μm as a reference. If thinner or thicker samples were measured, the MVTR was reported as corresponding to a 150 μm sample. Thus a 300 μm sample with a measured MVTR of 10 g/m²/24 h was reported as having MVTR=20 g/m²/24 h for a 150 μm sample because of the linear connection between thickness of sample and MVTR of sample.

Finally, we noted that by using this method, we introduced an error by using a supporting PU film. Utilizing the fact that the adhesive/film laminate was a system of two resistances in series eliminated the error. When the film and the adhesive are homogeneous, the transmission rate may be expressed as:

1/P(measured)=1/P(film)+1/P(adhesive).

Hence, by knowing the film permeability and thickness of the adhesive, it was possible to calculate the true permeability of the adhesive, P(adhesive), using the following expression:

P(adhesive)=d(adhesive)/150 μm*1/(1/P(measured)−1/P(film))

where d(adhesive) was the actual measured thickness of the adhesive and P(film) was the MVTR of the film without any adhesive on and P(measured) was the actual measured MVTR.

Moisture Absorption

An adhesive sheet of defined thickness was placed between two release liners. With a punching tool, samples were punched out. Sample size was 25×25 mm. The release liners were removed. The samples were glued to an object glass and placed in a beaker with physiological salt water and placed in an incubator at 37° C.

The sample was weighed at the outset (M(start)) and after 2 hours (M(2 hours). Before weighing, the object glass was dried off with a cloth. For a 25×25 mm sample the area was 6.25 cm² (the surface edges were left out of the area). The moisture absorption may be calculated as: Water absorption after 2 hours=(M(2 hours)−M(start))/6.25 cm². The result is in the unit g/cm² per 2 hours.

Peel

A sample of 25×100 mm was cut from the prepared 200 micrometer thick sheet composition and a 25×300 mm piece of auxiliary tape was then added on the top of the sample. After 30 minutes of conditioning at 23° C. and 50% relative humidity, the sample was mounted in a tensile testing machine (INSTRON 5564 from Instron) and a 90-degree peel test was carried out from a specified substrate at a speed of 304 mm/min. The results are given in N/25 mm.

The peel test was carried out in a climate-controlled room at 23° C. and 50% relative humidity. Peel angle was fixed at 90° and the peel speed was 304 mm/min. Dwell time was 30 minutes or 24 hours, as specified.

Substrate used was 127×30×2 mm Plexiglas XT, roughened by laser cutting in a 3D “Vector” pattern at 0.5 mm cuts.

The substrate mounted in steel plate was attached to the peel sledge. Adhesive strips were punched out from adhesive sheets with a specified thickness in the dimensions 25×100 mm. Auxiliary tape (25 mm width) was mounted on the adhesive with 10 mm overlap. The release liner was lifted in one end to make the overlap with the auxiliary tape. The adhesive was applied to the substrate by using an automatic roll with a load of 2 kg.

The average of the mean load was reported as N/25 mm. The failure type, i.e. cohesive or adhesive failure, was observed, recorded, and reported with the peel data.

For measuring peel force on adhesives activated by solvent, the sample is activated with activating solvent, e.g. wipe, and immediately placed on the substrate. Dwell time is specified and may, e.g., be 30 min, 2 hrs, or 4 hrs.

Erosion

The erosion of the composition/adhesive according to the invention is a function of the amount of hydrocolloid composition.

An adhesive sheet of defined thickness and an outer diameter of 55 mm and a hole of diameter of 25 mm was coated on the top surface with an impermeable low density polyethylene (LDPE) film.

The other side of the composition was attached to the surface of a Petri dish and the system was mounted in a 1 l jar in an upright position in the middle of the jar. The jar was half filled with 0.9% NaCl in demineralised water and closed with a lid. The jar was placed in a lying position between two rollers and was rolled with a speed of 20 rpm in one direction and 20 rpm in the other direction for each 1 minute. If erosion was seen as a result of missing material, this was noted. Also, the average swelling (in one side) in mm was measured. This result of this erosion measurement is an indication of the resistance to water. All data is measured after 24 hours. The test was done at 20° C.

### Example 1

Materials

BIO-PSA 7-4502 silicone adhesive from Dow Corning.

Caboxymethyl cellulose (CMC) Aquasorb A-800 PH hydrocolloid from Hercules.

Superior Potato Starch from KMC.

Silicone Fluid Q7-9180, 0.65 Centistokes (CST) from Dow Corning.

Compositions

Two adhesives were made (all parts are non-volatile w/w):

Recipe 1: 70% (w/w) BIO-PSA 7-4502 and 30% (w/w) CMC.

Recipe 2: 60% (w/w) BIO-PSA 7-4502, 20% (w/w) CMC, and 20% (w/w) potato starch.

For recipes 1 and 2 the specified amount of BIO-PSA 7-4502 was added to a 20 mL Speedmixer (Hauschild, model DAC 150.1 FVZ) container, followed by the specified amounts of CMC and potato starch. The mixture was mixed for 3 minutes at 3000 rpm.

### Experimental

This peel force experiment was designed to test whether an adhesive activated by exposure to solvent would exhibit an increased peel force as compared to the same adhesive without the activation. Both recipes were tested with and without activation.

A further parameter introduced was the dwell time. The dwell time is the time the adhesive sits on the substrate before being peeled. For the adhesives activated with solvent, a long dwell time means that a long time passes between the exposure to solvent and the peel measurement. Therefore, at the 24-hour dwell time, most or all of the solvent will have evaporated. On the other hand, some solvent may still be present after 30 minutes. For this reason, the 24-hour dwell time will provide an indication of whether or not the effect of the solvent on adhesion, measured as peel force, is maintained even after the actual solvent has evaporated.

For the experiment, the adhesives were coated onto a polyurethane film (30 micrometers, Intellicoat) using a Sheen film applicator to the required film thickness, and left to dry overnight in a fume cupboard. When dried, a release liner (Scotchpack 9755, 3M) was applied to the adhesive for protection until use. Hotmelt adhesives were pressed between two release liners, or between one release liner and one backing carrier film. Solvent based adhesives may also be coated onto release liner and then transferred to a backing carrier film.

The solvent used was silicone fluid Q7-9180 applied by use of a wet wipe comprising the silicone fluid.

Results

As seen in the table below, activation of the adhesive lead to significant increases in peel force for both tested adhesive compositions and for both tested dwell times. As illustrated clearly by the 24-hour dwell time, even when substantially all of the activating solvent has evaporated, the effect on peel force remains. In other words, the increase in peel force does not depend on the presence of the solvent in the adhesive, but can rather be seen as a lasting effect of the activation. While not wishing to be bound by theory, the present inventor hypothesizes that at least part of the explanation relates to the activated adhesive being better able to wet and cause anchoring to a rough substrate. Thus, the activated adhesive would be able to quickly form a strong and lasting bond to the substrate in a way the non-activated adhesive could not. And, as demonstrated by the 24-hour dwell time results, the initial strong bonding and anchoring leads to long-lasting strong adhesion, even when no or very little solvent remains in the adhesive.

| | Recipe 1 | | | | Recipe 2 | | | |
|---|---|---|---|---|---|---|---|---|
| | 30 min dwell time | | 24 hrs dwell time | | 30 min dwell time | | 24 hrs dwell time | |
| | Not | | Not | | Not | | Not | |
| | activated | Activated | activated | Activated | activated | Activated | activated | Activated |
| Peel force | 4.1 | 8.5 | 4.0 | 8.1 | 2.3 | 5.8 | 3.9 | 9.3 |
| (N/25 mm) | | | | | | | | |

In addition to the results relating to peel force, the following observations were made. On activation, and before the solvent has evaporated, a drop in moduli is observed. Also, on activation, and before solvent has evaporated, there is a drop in shear resistance. After solvent has evaporated, shear resistance is increased.

With hydrocolloid-containing recipes, there is significant absorption of water over time. Rate and amount of absorption depends on type and amount of hydrocolloids. BIO-PSA 7-4502 as such has insignificant absorption over time.

1-25. (canceled)

26. A method of attaching an adhesive base plate of an ostomy device to a skin surface of a user, comprising the steps of:
a) providing an adhesive base plate comprising a backing layer and an adhesive disposed on a skin-facing surface of the backing layer;
b) providing a solvent to at least partially dissolve the adhesive;
c) bringing the solvent into contact with the adhesive to provide a partially dissolved adhesive; and
d) applying the partially dissolved adhesive to the skin, thereby attaching the adhesive base plate to the skin surface of the user.

27. The method according to claim 26, wherein the method includes a further step of applying the solvent or a second solvent to the skin surface of the user.

28. The method according to claim 26, wherein step (c) comprises applying the solvent to the adhesive.

29. The method according to claim 26, wherein the adhesive base plate further comprises a release liner disposed on the skin-facing surface of the adhesive, and wherein the method comprises the further step of removing the release liner from the adhesive, performed prior to step (c).

30. The method according to claim 26, wherein step (c) occurs before step (d).

31. The method according to claim 26, wherein step (c) occurs after step (d).

32. The method according to claim 26, wherein step (c) occurs at the same time as step (d).

33. The method according to claim 26, wherein the adhesive comprises a polymer selected from the group consisting of silicone, styrene block copolymer, polyurethane, acrylate, ethylene block copolymer, styrene-isoprene-styrene (SIS), ethylene vinyl acetate (EVA), styrene-butadiene-styrene (SBS), butyl rubber, polyisobutylene (PIB), polyether, polyester, polyurea, and epoxy-based polymer.

34. The method according to claim 26, wherein the solvent is selected from the group consisting of silicone fluid, hexamethyldisiloxane (HMDS), heptane, ethyl acetate, acetone, tetrahydrofuran (THF), butyl acetate, hexane, ethanol, iso-propanol, dichloromethane, methanol, acetonitrile, toluene, dimethylformamide (DMF), dimethyl sulfoxide (DMSO), pyridine, cyclohexane, and n-butanol.

35. The method according to claim 26, wherein the solvent is applied to the skin and/or the adhesive by a method selected from the group consisting of wiping, spraying, painting, dripping, rolling, squirting, brushing, or dipping.

36. The method according to claim 26, wherein the adhesive has a higher tack after having been brought into contact with the solvent in comparison to the tack prior to contact with the solvent.

37. The method according to claim 26, wherein the adhesive has a higher peel force after having been brought into contact with the solvent in comparison to the peel force prior to contact with the solvent.

38. The method according to claim 26, wherein the adhesive has a lower shear force after having been brought into contact with the solvent in comparison to the shear force prior to contact with the solvent.

39. The method according to claim 26, wherein the adhesive has a lower viscosity after having been brought into contact with the solvent in comparison to the viscosity prior to contact with the solvent.

40. The method according to claim 26, wherein the adhesive has a lower modulus after having been brought into contact with the solvent in comparison to the modulus prior to contact with the solvent.

41. The method according to claim 26, wherein the adhesive has a lower cohesion after having been brought into contact with the solvent in comparison to the cohesion prior to contact with the solvent.

42. The method according to claim 26, wherein the adhesive base plate comprises one or more release liners, and wherein the method comprises the further step of:
a1) removing one or more releaser liners.

43. The method according to claim 42, wherein the adhesive base plate comprises two or more release liners, and wherein at least one release liner is removed prior to step (a) and at least one release liner is removed between steps (c) and (d), or after step (d).

44. The method according to claim 43, comprising the sequential steps of:
a) providing the adhesive base plate comprising the backing layer and the adhesive disposed on the skin-facing surface of the backing layer;
a1) removing one or more release liners;
b) providing the solvent to partially dissolve the adhesive;
c) bringing the solvent into contact with the adhesive exposed by removal of the one or more release liners in step (a1) to provide partially dissolved and exposed adhesive;
c1) removing one or more release liners; and
d) applying the partially dissolved and exposed adhesive of step c) to the skin, thereby attaching the adhesive base plate to the skin surface of the user.

45. The method according to claim 43, comprising the sequential steps of:
a) providing the adhesive base plate comprising the backing layer and the adhesive disposed on the skin-facing surface of the backing layer;
a1) removing one or more release liners;
b) providing the solvent to partially dissolve the adhesive;
c) bringing the solvent into contact with the adhesive exposed by removal of the one or more release liners in step (a1) to provide partially dissolved and exposed adhesive;
d) applying the partially dissolved and exposed adhesive of step c) to the skin, thereby attaching the adhesive base plate to the skin surface of the user;
d1) removing one or more release liners;
b1) providing the solvent to partially dissolve the adhesive protected by the release liners of step d1);
c1) bringing the solvent into contact with the adhesive exposed by removal of the one or more release liners is step d1) to provide partially dissolved and exposed adhesive; and
d2) applying the partially dissolved and exposed adhesive in step (c1) to the skin surface of the user.

46. A kit comprising an adhesive base plate for an ostomy device, the adhesive base plate comprising an adhesive, and the kit further comprising a solvent to partially dissolve the adhesive.

47. The kit according to claim 46, wherein the adhesive is an adhesive according to claim 8.

48. The kit according to claim 46, wherein the solvent is a solvent according to claim 9.

49. The kit according to claim 46, wherein the kit further comprises instructions for use.

50. The kit according to claim 46, wherein the adhesive base plate comprises two or more release liners.
